(19) United States
(12) Patent Application Publication
(10) Pub. No.: US 2004/0044399 A1
(43) Pub. Date: Mar. 4, 2004

(54) Radiopaque links for self-expanding stents

(76) Inventor: Joseph A. Ventura, Salinas, CA (US)

Correspondence Address: FULWIDER PATTON LEE & UTECHT, LLP, HOWARD HUGHES CENTER, 6060 CENTER DRIVE, TENTH FLOOR, LOS ANGELES, CA, 90045, US

(21) Appl. No.: 10/235,248

(22) Filed: Sep. 4, 2002

(51) Int. Cl. A61F 2/06

(52) U.S. Cl. 623/1.16; 623/1.34

(57) ABSTRACT

A longitudinally flexible stent having high visibility under fluoroscopy for implanting in a body lumen. The stent includes a plurality of radiopaque interconnecting elements, configured to be expandable and encapsulated by polymeric material in a coil-like configuration, that are connected to a plurality of cylindrical elements at least at distal and proximal ends of the stent such that the radiopaque interconnecting elements are visible under fluoroscopy to enable identification of the position, diameter, and length of the stent at the implantation site. Alternatively, the stent includes a plurality of radiopaque interconnecting elements having a polymeric material filled with a radiopaque material attached to the cylindrical elements of the stent at least at the distal and proximal ends of the stent.

### BACKGROUND OF THE INVENTION

This invention generally relates to expandable endoprosthesis devices, often referred to as stents, and more particularly, to the radiopaque marking of such devices.

Stents are useful in the treatment and repair of atherosclerotic stenosis in blood vessels and are generally cylindrically-shaped devices which function to hold open a segment of a blood vessel or other arterial lumen, such as a coronary artery. Stents are usually delivered in a compressed condition to the target site and then deployed at that location into an expanded condition to support the vessel and help maintain it in an open position. They are particularly suitable for use in supporting and holding back a dissected arterial lining which could otherwise occlude the fluid passageway therethrough.

In order to accomplish precise placement of stents, various means are employed to identify the position of the stent within a blood vessel. One means frequently described for accomplishing precise placement of a stent is the attachment of radiopaque markers to the stent so that through the use of fluoroscopy, the position of the stent within a blood vessel can be identified. Radiopaque markers are partially needed when the stent is made from nickel-titanium alloy, which has low visibility on a fluoroscope. Once the stent with its radiopaque markers has been implanted, subsequent checkups of the treated segment are easily performed since the markers remain visible under fluoroscopic illumination.

Some conventional radiopaque markers, however, have a number of limitations. Upon attachment to a stent, conventional radiopaque markers may project from the surface of the stent, thereby comprising a departure from the ideal profile of the stent. Such conventional radiopaque markers may protrude from the walls of the stent and depending upon their location upon the stent, may either project inwardly to disrupt blood flow therethrough or outwardly to traumatize the walls of the blood vessel. In addition, conventional radiopaque markers have the disadvantage in that their attachment to the stent can be tedious and imprecise. Moreover, the configuration of many heretofore known markers fail to provide a precise indication of the location and position of the stent. Finally, galvanic corrosion might result from the contact of two disparate metals, i.e., the metal used in the construction of the stent and the radiopaque metal of the marker which could eventually cause the marker to become separated from the stent. Such an occurrence would be problematic should the marker embolize downstream and occlude the artery.

Other conventional radiopaque markers restrict the expansion capabilities of an expandable stent by adding rigidity to the stent in areas designated for stent deformation. Still other conventional stents utilize material, such as tantalum, that is effective for use in identifying the location of a stent within a vessel, but fluoroscopically illuminates so brightly so as to obscure proper visibility of the arterial lesion, possibly impairing the ability to repair the lesion. Finally, some conventional radiopaque markers do not generally, under fluoroscopy, provide the operator with means to accurately assess stent length and diameter.

Stents also have been previously marked by plating selected portions thereof with a radiopaque material. An advantageously selected pattern of plated areas would theoretically allow the position, length and diameter of the stent to be discerned. However, due to the minimal thickness of the plating, sometimes only an extremely faint fluoroscopic image can be generated which may ultimately limit its utility.

To overcome the problems and limitations associated with stents having conventional radiopaque markers, or plated markings, it would be desirable to employ radiopaque markers or markings that do not limit the expansion capabilities of an expandable stent, nor alter the profile of the stent. Such markers should be clearly visible, provide means to assess stent length and diameter and do not obscure the blood vessel lesion being repaired. Such markers should not be detrimentally affected by galvanic corrosion. The present invention satisfies these and other needs.

### SUMMARY OF THE INVENTION

The present invention provides for a radiopaque marker in the form of a radiopaque interconnecting element or link of a stent that effectively identifies the position, diameter and length of the stent both while attached to the delivery device as well as upon implantation within a blood vessel. The present invention does this without obscuring the lesion being repaired. The radiopaque interconnecting element is formed as an integral part of the stent in that it should not protrude from the surface of the stent and does not limit the expansion capabilities of the stent. Furthermore, the radiopaque interconnecting element is not adversely affected by galvanic corrosion. The radiopaque interconnecting element of the present invention may be utilized with stents having various geometric shapes and materials. The stent is formed of a biocompatible material, such as stainless steel, tungsten, tantalum, superelastic nickel titanium alloys, and thermal plastic polymers. In addition, the radiopaque interconnecting elements may be positioned anywhere on the stent and any acceptable means for attaching the radiopaque interconnecting elements to the stent may be employed. It is impartial, however, that the means for attaching the radiopaque interconnecting element, its location within the stent, and the material and geometric shape of the stent, be selected so that a stent incorporating the radiopaque interconnecting element of the present invention may benefit from the advantages provided herein.

In one embodiment, the present invention consists of a longitudinally flexible stent having high visibility under fluoroscopy for implanting in a body lumen. The stent includes a plurality of adjacent cylindrical elements with each cylindrical element having a circumference extending about a longitudinal stent axis and being substantially independently expandable in the radial direction. The plurality of adjacent cylindrical elements are arranged in alignment along the longitudinal stent axis and form a generally serpentine wave pattern transverse to the longitudinal axis while containing a plurality of alternating valley portions and peak portions. The stent further includes a plurality of interconnecting elements extending between the adjacent cylindrical elements and connecting the adjacent cylindrical elements to one another. For example, the plurality of cylindrical elements can be formed from a tubular member or a flat sheet of material. In one aspect of the present invention, a plurality of radiopaque interconnecting elements are configured to be expandable while encapsulated by polymeric material. The plurality of radiopaque interconnecting elements are connected to the cylindrical elements at least at distal and proximal ends of the stent so that the radiopaque interconnecting elements are visible under fluoroscopy and the respective distal and proximal stent ends can be easily located in the body lumen at the implantation site. Alternatively, in order to maintain rigidity of the links at each stent end, the radiopaque interconnecting elements can be alternated with non-radiopaque links at the distal and proximal ends of the stent.

In another aspect of the present invention, the radiopaque interconnecting elements are shaped in a coiled configuration and encapsulated by a polymeric shell. Various metals that can be used in forming the coiled radiopaque interconnecting elements include gold, platinum, tantalum, and platinum/10% iridium. The polymeric material encapsulating the interconnecting elements can be any biocompatible polymer, such as urethane. The thickness and length of the radiopaque-coiled interconnecting element is dictated by the particular stent design used.

In an alternative embodiment, the plurality of radiopaque interconnecting elements are connected to the cylindrical elements throughout the length of the stent so that the radiopaque interconnecting elements are visible under fluoroscopy and the stent can be easily located in the body lumen at the implantation site.

In another aspect, the present invention consists of a longitudinally flexible stent having high visibility under fluoroscopy for implanting in a body lumen. The stent includes a plurality of adjacent cylindrical elements with each cylindrical element having a circumference extending about a longitudinal stent axis and being substantially independently expandable in the radial direction. The plurality of adjacent cylindrical elements are arranged in alignment along the longitudinal stent axis and form a generally serpentine wave pattern transverse to the longitudinal axis containing a plurality of alternating valley portions and peak portions. The present invention further includes a plurality of interconnecting elements that extend between adjacent cylindrical elements and connect adjacent cylindrical elements to one another. A plurality of radiopaque interconnecting elements have a polymeric material filled with a radiopaque material attached to the cylindrical elements of the stent. In addition, the plurality of radiopaque interconnecting elements are connected to the cylindrical elements at least at distal and proximal ends of the Patent so that the radiopaque material is visible under fluoroscopy and the distal and proximal stent ends can be easily located in the body lumen at the implantation site.

The plurality of interconnecting elements are adapted to assume a number of various configurations including linear, non-linear, or VTS arrangements. Again, the plurality of cylindrical elements can be formed from a tubular member or a flat sheet of material. Various biocompatible materials that can be used in forming the stent include stainless steel, tungsten, tantalum, superelastic nickel titanium alloys, shape memory alloy materials, pseudoelastic alloy materials, and thermal plastic polymers. The polymeric material can be any biocompatible material such as urethane, and the radiopaque material, which fills the polymeric material, can be either tungsten or barium sulfate. Again, the thickness and length of the radiopaque-filled polymeric material, interconnecting member varies depending on the particular stent design used.

In one embodiment, the radiopaque-filled polymeric material is in communication with the interconnecting element of the invention to form the radiopaque interconnecting element and is attached thereto by micro-injection molding.

In an alternative embodiment, the plurality of radiopaque interconnecting elements are connected to the cylindrical elements throughout the stent so that the radiopaque interconnecting elements are visible under fluoroscopy and the stent can be easily located in the body lumen at the implantation site.

Other features and advantages of the present invention will become more apparent from the following detailed description of the invention when taken in conjunction with the accompanying exemplary drawings.

### BRIEF DESCRIPTION OF THE DRAWINGS

FIG. 1 is an elevational view, partially in section, depicting a stent embodying features of the present invention mounted on a delivery catheter disposed within a vessel.

FIG. 2 is an elevational view, partially in section, similar to that shown in FIG. 1, wherein the stent is expanded within a vessel, pressing the lining against the vessel wall.

FIG. 3 is an elevational view, partially in section, showing the expanded stent within the vessel after withdrawal of the delivery catheter.

FIG. 4A is a partial flattened view of the stent embodying radiopaque interconnecting elements attached to cylindrical elements at distal and proximal ends of the stent.

FIG. 4B is a partial flattened view of the stent embodying radiopaque alternate interconnecting elements attached to a plurality of cylindrical elements throughout the entire length of the stent.

FIG. 4C is an enlarged plan view of the flattened stent of FIG. 4A depicting an individual radiopaque interconnecting element encapsulated by a polymeric shell.

FIG. 5 is a schematic view of the embodiment of FIG. 4A having radiopaque interconnecting elements attached to adjacent cylindrical elements at the distal and proximal stent ends while the stent is in an expanded configuration.

FIG. 6 is a schematic view of the alternative embodiment of FIG. 4B having radiopaque alternate interconnecting elements attached to the plurality of cylindrical elements throughout the entire length of the stent while the stent is in an expanded configuration.

FIG. 7A is a partial flattened view of an alternative embodiment of the present invention depicting radiopaque-filled, polymer interconnecting elements in accordance with the present invention.

FIG. 7B is an enlarged plan view of the flattened stent of FIG. 7A depicting an individual radiopaque-filled, polymeric interconnecting element.

FIG. 8 is a schematic view of the stent of FIG. 7A embodying radiopaque-filled, polymer interconnecting elements attached to adjacent cylindrical elements at the distal and proximal stent ends while the stent is in an expanded configuration.

FIG. 9 is a schematic view of the stent of FIG. 7A embodying additional alternating radiopaque-filled, polymer interconnecting elements attached to a plurality of cylindrical elements throughout the entire length of the stent while the stent is in an expanded configuration.

### DETAILED DESCRIPTION OF THE PREFERRED EMBODIMENTS

The stent of the present invention includes modified radiopaque markings in the form of interconnecting elements or links that render the position of the stent clearly visible without obscuring the image of the treatment site within a body lumen. This enables the position of the stent to be monitored as it is being advanced through the vasculature by the delivery catheter. Accordingly, the unique design of the radiopaque links allows the stent to be very precisely positioned relative to the target site, and further allows its deployment to be verified and its continued presence to be detected at any time thereafter.

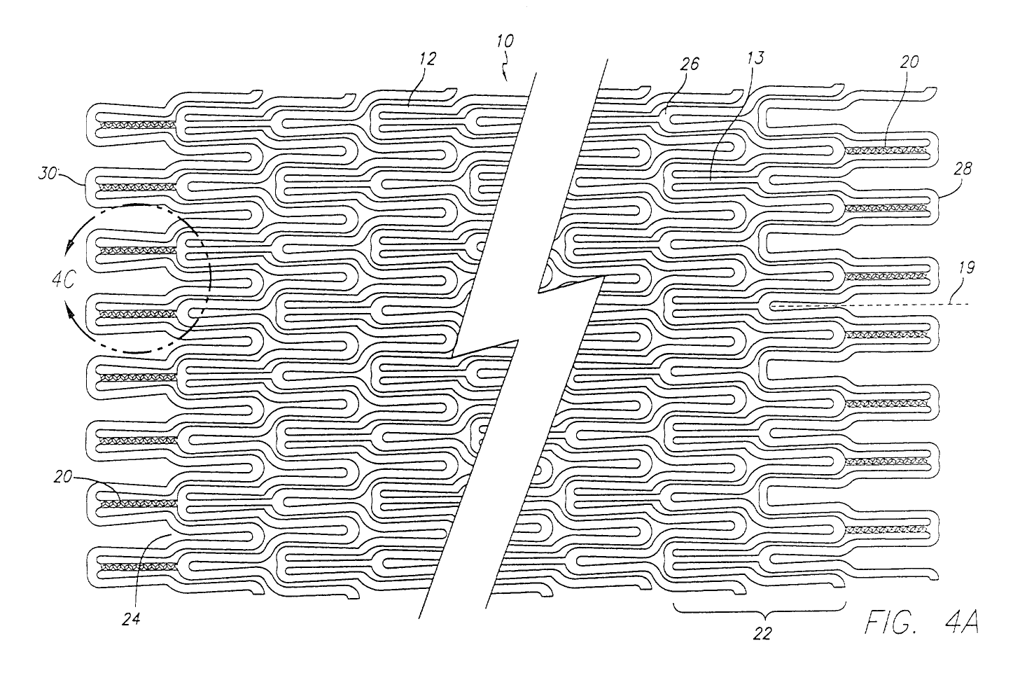

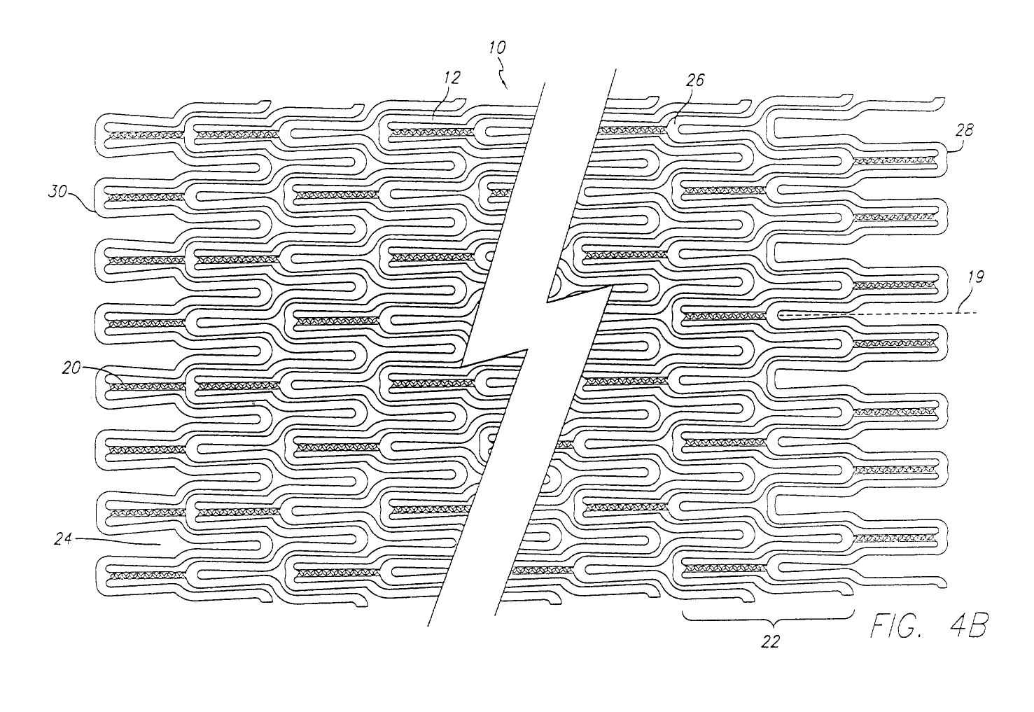

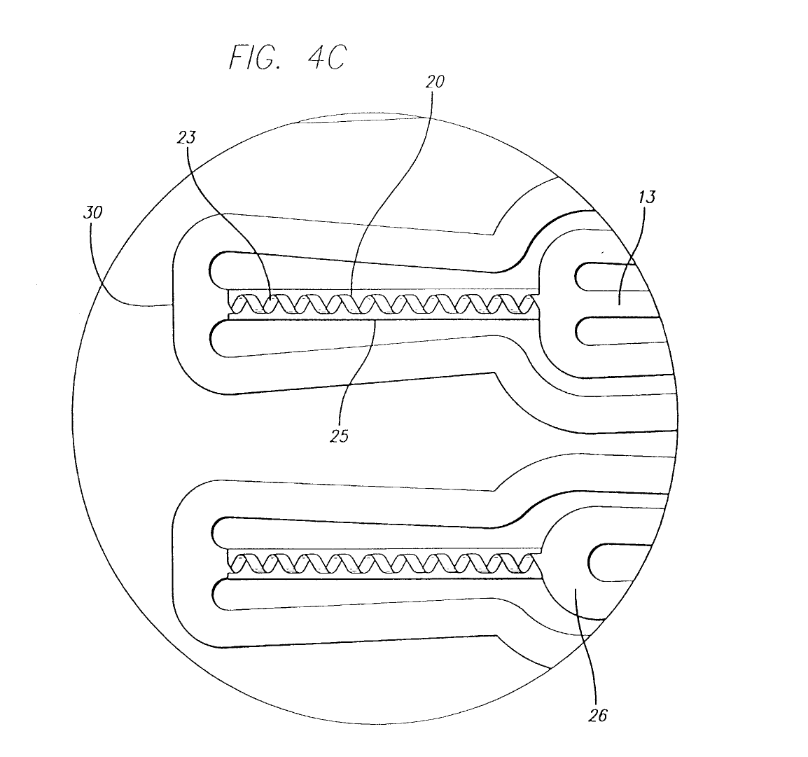

With reference to the drawings, FIG. 1 illustrates an exemplary embodiment of stent 10 incorporating features of the present invention, which stent is mounted onto delivery catheter 11. FIG. 4 is a plan view of this exemplary embodiment stent 10 with the structure flattened out into two dimensions in order to facilitate explanation. Stent 10 generally comprises a plurality of radially expandable cylindrical elements 12 disposed generally coaxially and interconnected by interconnecting elements 13 disposed between adjacent cylindrical elements 12. The delivery catheter 11 has an inner tubular member 14 upon which the collapsed stent 10 is mounted. A restraining sheath 15 extends over both the inner tubular member 14 and stent 10 in a co-axial relationship. The stent delivery catheter 11 is used to position the stent 10 within an artery 16 or other vessel. The artery 16, as shown in FIG. 1, has a dissected or detached lining 17 which has occluded a portion of the arterial passageway.

In one embodiment, the delivery of the stent 10 is accomplished in the following manner. Stent 10 is first mounted onto the delivery catheter 11 with the restraining sheath placed over the collapsed stent. The catheter-stent assembly can be introduced within the patient's vasculature in a conventional Seldinger technique through a guiding catheter (not shown). A guide wire 18 is disposed through the damaged arterial section with the detached or dissected lining 17. The catheterstent assembly is then advanced over guide wire 18 within artery 16 until the stent 10 is directly under the detached lining 17. The restraining sheath 15 is retracted exposing the stent 10 and allowing it to expand against the inside of artery 16, which is illustrated in FIG. 2. While not shown in the drawing, artery 16 is preferably expanded slightly by the expansion of stent 10 to seat or otherwise embed stent 10 to prevent movement. Indeed, in some circumstances during the treatment of stenotic portions of an artery, the artery may have to be expanded considerably in order to facilitate passage of blood or other fluid therethrough.

While FIGS. 1-3 depict a vessel having detached lining 17, stent 10 can be used for purposes other than repairing the lining. Those other purposes include, for example, supporting the vessel, reducing the likelihood of restenosis, or assisting in the attachment of a vascular graft (not shown) when repairing an aortic abdominal aneurysm.

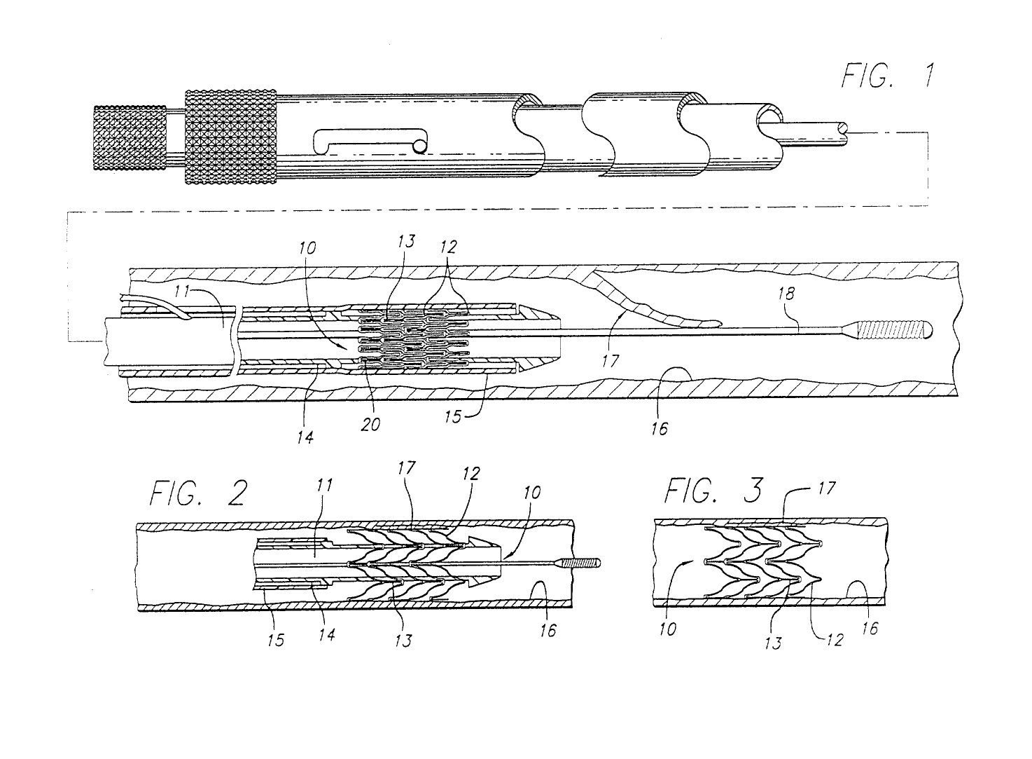

In general, stent 10 serves to hold open the artery 16 after catheter 11 is withdrawn, as illustrated in FIG. 3. Due to the formation of stent 10, the undulating component of the cylindrical elements of stent 10 is relatively flat in a transverse cross-section so that when stent 10 is expanded, cylindrical elements 12 are pressed into the wall of artery 16, and, as a result, do not interfere with the blood flow through artery 16. Cylindrical elements 12 of stent 10 that are pressed into the wall of artery 16 will eventually be covered with endothelial cell growth that further minimizes blood flow turbulence. The serpentine pattern of cylindrical sections 12 provide good tacking characteristics to prevent stent movement within the artery. Furthermore, the closely spaced cylindrical elements 12 at regular intervals provide uniform support for the wall of artery 16, and consequently are well adapted to tack up and hold in place small flaps or dissections in the wall of artery 16 as illustrated in FIGS. 2 and 3.

As is shown in the following drawings, which are included for purposes of illustration and not by way of limitation, the invention is embodied in a modified radiopaque marker in the form of interconnecting elements or links 20 (FIGS. 4-9). Conventional radiopaque markers are limited in that they may comprise undesirable projections extending from a stent, may be arduous to attach, restrict the expansion capabilities of an expandable stent and may be ineffective in the identification of the position, orientation and configuration of a stent. The radiopaque interconnecting elements of the present invention define an acceptable, very low profile, and may be conveniently affixed to a stent through micro-injection molding which do not impede the expansion capabilities of an expandable stent. As such, the markers help to identify the position, orientation and configuration of a stent within a blood vessel. Thus, the radiopaque interconnecting elements provide superior means for locating the position of the stent in a body lumen at the implantation site.

The present invention facilitates precise placement of a stent 10 by way of its novel configuration, position upon a stent, and material properties. The characteristics of radiopaque interconnecting elements 20 are selected to assure that a stent embodying the radiopaque interconnecting element may benefit from the advantages which the invention provides. Thus, the radiopaque interconnecting elements may have various geometric shapes, comprise various materials and may be positioned anywhere on a stent so long as the desired advantages of the invention are achieved. The interconnecting elements of the present invention have a wide range of vascular applications, including the areas of coronary, peripheral (iliacs, SFA, and carotids), and AAA implants.

While stent 10 can include any number of configurations as shown in the following figures, one embodiment of the present invention includes a longitudinally flexible stent having high visibility under fluoroscopy for implanting in a body lumen 21. As shown in FIG. 4A, the stent consists of a plurality of cylindrical elements 12 with each cylindrical element having a circumference extending about a longitudinal stent axis 19 and being substantially independently expandable in the radial direction. The plurality of adjacent cylindrical elements are arranged in alignment along the longitudinal stent axis and form a generally serpentine wave pattern 22 transverse to the longitudinal stent axis. This characteristic serpentine wave pattern of the plurality of adjacent cylindrical elements consists of a plurality of alternating valley portions 24 and peak portions 26. A plurality of interconnecting elements 13 extend between the adjacent cylindrical elements and connect the adjacent cylindrical elements to one another. Stent further includes a plurality of radiopaque interconnecting elements 20 configured to be expandable and formed of a coiled radiopaque material 23 encapsulated by a polymeric shell 25. FIGS. 4A-4B depict these interconnecting elements 20 connected to the cylindrical elements at least at both distal and proximal ends 28 and 30 of the stent so that the radiopaque material is visible under fluoroscopy and the distal and proximal stent ends can be easily located in the body lumen at the implantation site.

Alternatively, as shown in FIG. 4B of the present invention, stent 10 can have a plurality of radiopaque interconnecting elements 20 interspersed throughout its entire length including at both distal and proximal stent ends 28 and 30. It should be appreciated that the present invention contemplates the placement of radiopaque interconnecting elements in any number of different configurations, and is not limited to the configuration shown in FIG. 4B. Such an arrangement of radiopaque interconnecting elements throughout the entire stent length allows the physician to observe each cylindrical element under fluoroscopy during deployment of stent or during a follow up intervention. FIG. 4C depicts an enlarged, up close view of the proximal stent end connected to an adjacent ring by the radiopaque interconnecting element.

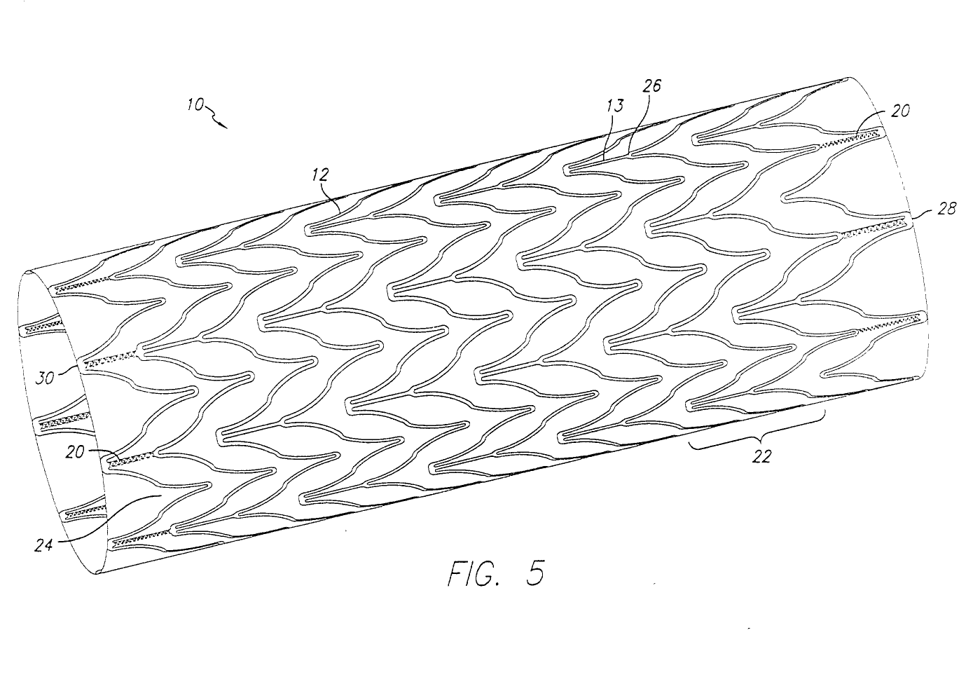

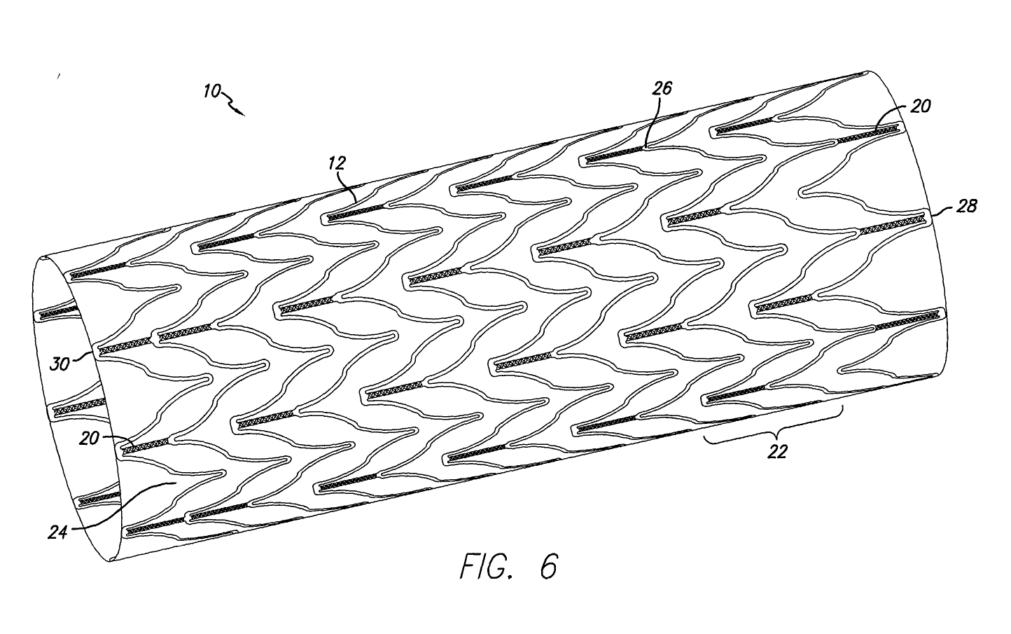

FIG. 5 illustrates one embodiment of the present invention of FIG. 4A while the stent is in an expanded configuration. The stent 10 includes the plurality of interconnecting elements 20 attached to the cylindrical elements 12 at each of the distal and proximal stent ends 28 and 30. This “W” pattern at the distal and proximal stent ends helps increase the overall radiopacity as well as the flexibility and strength of the stent at each of the stent ends by virtue of the radiopaque coil encapsulated by the polymer shell 25. As a result, the stent should be easily observable by a physician using imaging instrumentation, such as a fluoroscope.

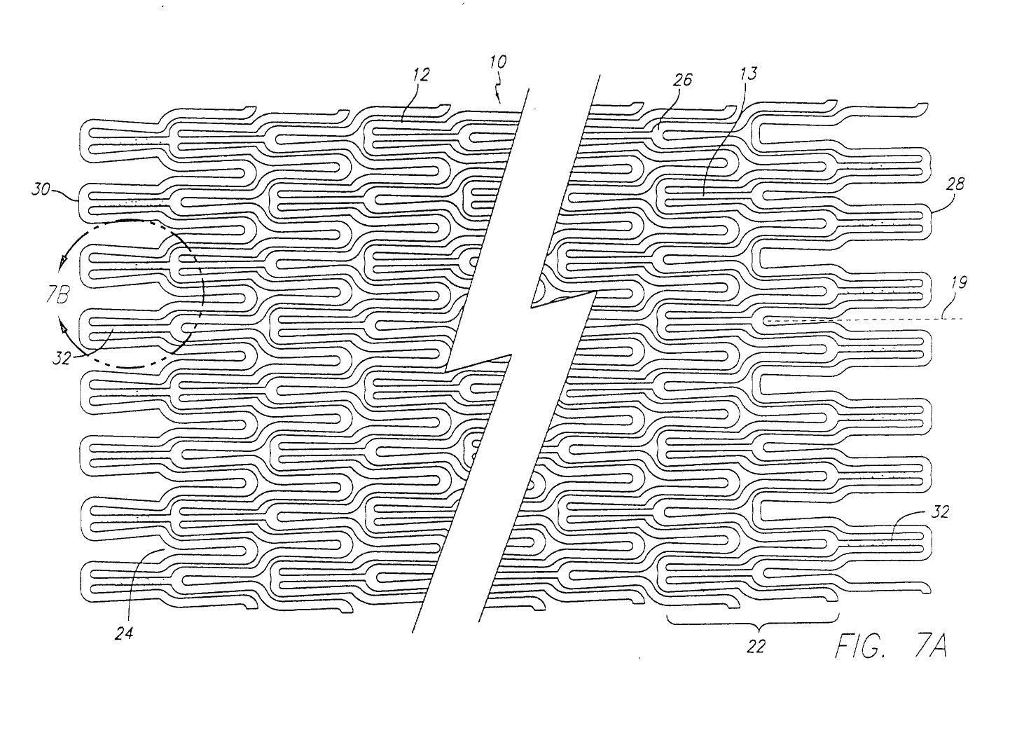

FIG. 6 illustrates the alternative embodiment of FIG. 4A while the stent is in an expanded configuration. The stent 10 consists of the plurality of radiopaque interconnecting elements 20 attached to adjacent cylindrical elements 12 throughout the entire length of the stent. Again, this particular type of arrangement of the radiopaque interconnecting elements greatly enhances the overall radiopacity of the stent. The attachment of the radiopaque interconnecting elements to each of the adjacent cylindrical elements throughout the body of the stent also helps increase the flexibility of the stent and prevents the shortening of the stent during radial expansion.

Essentially any biocompatible polymer material can be used in conjunction with the radiopaque coil 23 of the radiopaque interconnecting elements 20. Exemplary of one such polymer that can be used in accordance with the invention is urethane. In addition, it is important that the biocompatible polymer material possesses other important physical properties, such as the ability to adhere well to the stent matrix, and the ability to withstand stent integrity testing (i.e., corrosion, and accelerated fatigue testing). The polymeric encapsulation 25 of the radiopaque coiled interconnecting element protects the nitinol stent body from corrosive interaction with the radiopaque coil material.

With further reference to FIGS. 4-6 of the present invention, various radiopaque materials that can be used in forming the coil 23 of the radiopaque interconnecting elements 20 include gold, platinum, tantalum, and platinum/10% iridium. The coil of the radiopaque interconnecting elements is typically manufactured with a rectangular flatwire or use of a round wire. The primary function of the coil structure is to provide flexibility and radiopacity for the stent 10. Additional features, such as column strength can be improved to minimize stent shortening or assist in stent loading into the delivery system. This can be accomplished by modifying the cross section and/or the pitch of the coil. For example, by changing the cross-sectional area of the coil, the performance properties of the radiopaque interconnecting elements, such as bending, flexibility and rigidity, can be modified. Generally, increasing the width and/or thickness of the flatwire coil, or increasing the diameter of the round wire, adds more strength and rigidity to the coil, but detracts from its flexibility. Further, increasing the pitch (i.e., the distance between adjacent coil rings) of the coil causes the coil to take on more flexibility. In order to maintain rigidity of the coil, the pitch can be minimized in such a manner so that all of the coil rings are in contact with each other.

While not shown in FIGS. 4-6, it should also be appreciated that the present invention further contemplates a configuration consisting of a plurality of non-radiopaque links alternating with the plurality of radiopaque coiled interconnecting elements 20 at both ends of the stent in order to maintain rigidity of the links at the stent ends.

The polymeric encapsulated, radiopaque coiled interconnecting elements 20 of the present invention are attached to the plurality of adjacent cylindrical elements 12 of the stent through use of micro-injection molding, a process well known in the art. Alternatively, the radiopaque coiled elements can be attached to adjacent cylindrical elements of a particular stent design by one of the well known processes in the art of micro-welding and resistance welding the interconnecting elements to the base pattern. The use of a mechanical junction is yet another alternative for connecting the radiopaque coiled interconnecting members to adjacent cylindrical elements of the stent which can be directly designed into the particular stent pattern.

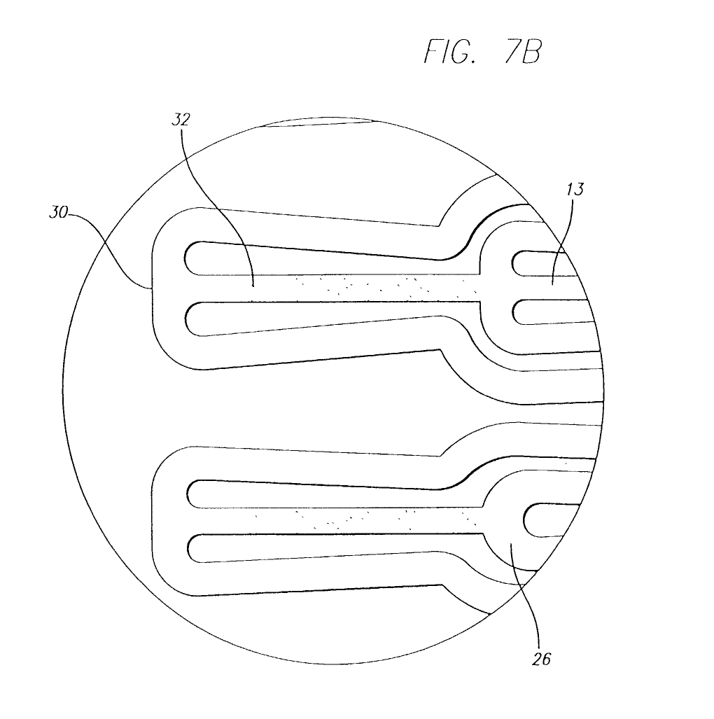

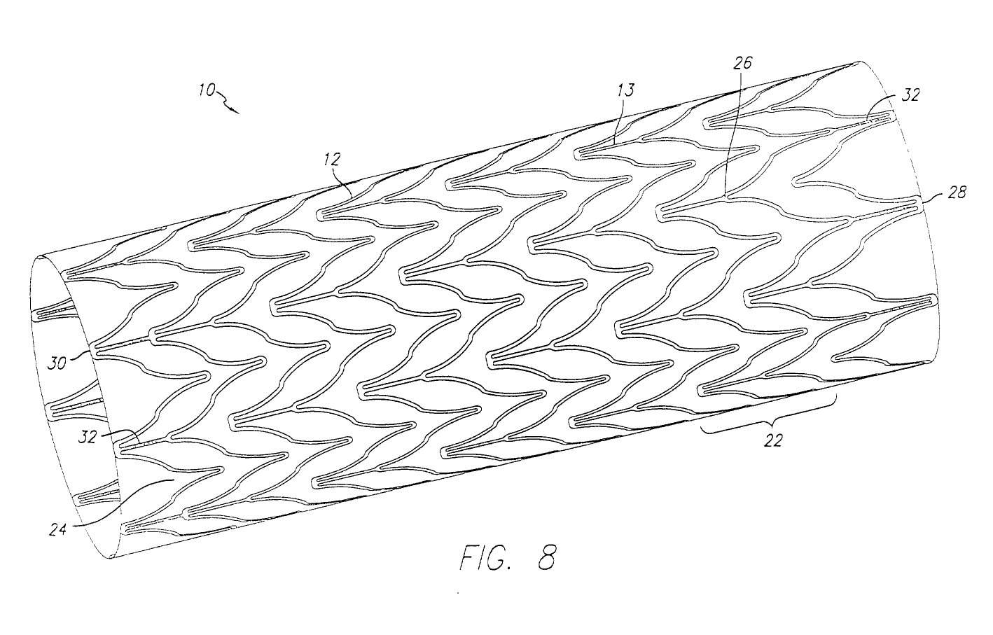

In another embodiment of the present invention as shown in FIG. 7A, a longitudinally flexible stent 10 having high visibility under fluoroscopy for implanting in a body lumen 21 includes a plurality of adjacent cylindrical elements 12 with each cylindrical element having a circumference extending about a longitudinal stent axis 19 and being substantially independently expandable in the radial direction. The plurality of adjacent cylindrical elements 12 are arranged in alignment along the longitudinal stent axis and form a generally serpentine wave pattern 22 transverse to the longitudinal stent axis. A plurality of alternating valley portions 24 and peak portions 26 form the characteristic serpentine wave pattern of the stent. Adjacent cylindrical elements are connected to one another by a plurality of interconnecting elements 13 which extend between each adjacent cylindrical element throughout the stent. Further, a plurality of radiopaque interconnecting elements 32 having a polymeric material filled with a radiopaque material are attached to the cylindrical elements of the stent at least at distal and proximal ends 28 and 30 of the stent so that the radiopaque interconnecting elements are visible under fluoroscopy and the distal and proximal stent ends can be easily located in the body lumen at the implantation site. FIG. 7B illustrates an enlarged, up close view of an individual radiopaque-filled, polymeric interconnecting element of the stent.

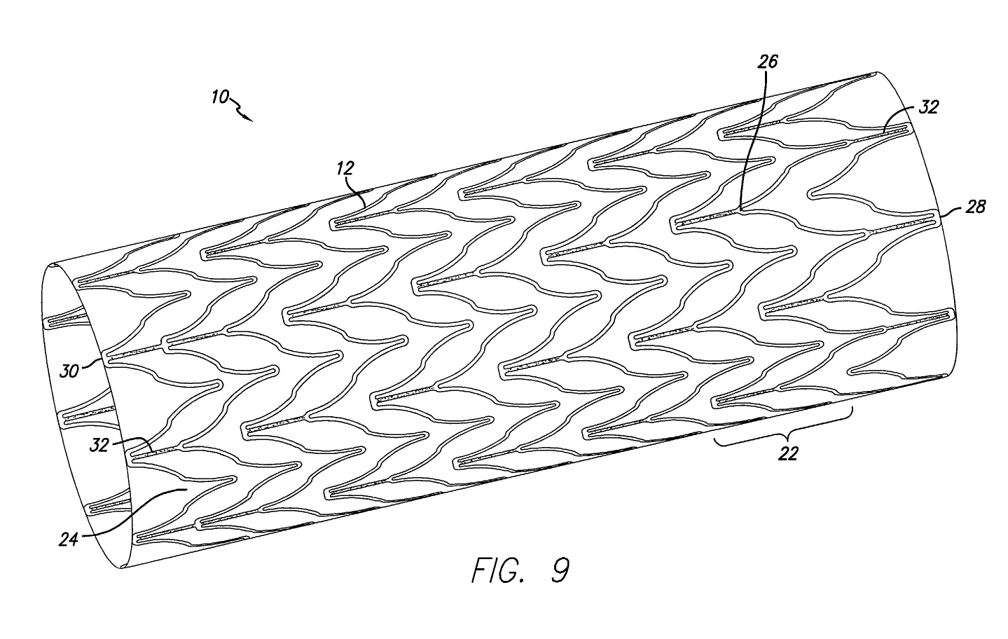

FIG. 8 shows the embodiment of the stent of FIG. 7A while the stent is in an expanded configuration. Specifically, the placement of the radiopaque interconnecting elements 32 at the distal and proximal stent ends 28 and 30 helps provide increased radiopacity at each stent end for viewing under fluoroscopy, and such other desirable attributes, including flexibility and increased strength at the ends of the stent.

Alternatively, as shown in FIG. 9 of the present invention, the plurality of radiopaque-filled polymeric interconnecting elements 32 are connected to adjacent cylindrical elements 12 throughout the entire length of the stent so that the radiopaque interconnecting elements are visible under fluoroscopy and the stent can be easily located in the body lumen at the implantation site. The present invention contemplates the use of radiopaque interconnecting elements in any number of different configurations, and is not limited to the configuration shown in FIG. 9.

It should be appreciated that the radiopaque-filled polymeric interconnecting elements can be configured to assume a variety of different arrangements, such as a linear, non-linear or a VTS configuration, among others. However, as illustrated in FIGS. 7-9, the alternate interconnecting elements 32 have a linear configuration but are by no means limited to such arrangement.

With further reference to FIGS. 7-9 of the present invention, various radiopaque filler materials that can be used in combination with the polymer-based, radiopaque interconnecting elements 32 include barium sulfate and tungsten.

Essentially any biocompatible polymer material can be used in conjunction with the radiopaque filler material of the radiopaque interconnecting elements 32. Exemplary of one such polymer that may be used in accordance with the invention is urethane. In addition, it is important that the biocompatible polymer material possesses other important physical properties, such as the ability to adhere well to the stent matrix, and the ability to withstand stent integrity testing (i.e., corrosion, and accelerated fatigue testing).

In keeping with the invention, the radiopaque-filled polymeric interconnecting elements 32 of the second embodiment are fabricated so that the radiopaque filler has an increased durometer (i.e., by increasing the stiffness of the radiopaque filler material) in order to compensate for having no supportive radiopaque coil 23 inside of the radiopaque interconnecting element. The polymeric radiopaque interconnecting elements 32 of the present invention are attached to the plurality of adjacent cylindrical elements of the stent through use of micro-injection molding, a process well known in the art. The surface of the polymer can be loaded with a variable amount of radiopaque filler material depending on the desired radiopaque intensity.

The incorporation of the radiopaque-filled, polymeric interconnecting elements 32 into the design of various types of stents afford such stents many advantages. For example, the unique design of the interconnecting elements provide the particular stent with improved radiopacity, maintained device attributes from the base stent design, and no metallic interaction due to the use of dissimilar metals (i.e., exposed marker bands).

It is further contemplated by the present invention that both types of radiopaque interconnecting elements (the coil 23 and radiopaque-filled polymeric interconnecting elements) 20 and 32 can be selectively placed on low stress areas (i.e. a strut) of the stent.

The thickness and length of the radiopaque interconnecting elements (both the coil 23 and radiopaque-filled polymeric interconnecting elements) 20 and 32 are dictated by the particular stent design used. The figures set forth herein embody the particular stent design of the DYNALINK™ stent manufactured by Advanced Cardiovascular Systems, Inc., Santa Clara, Calif. The structural details of the DYNALINK™ stent are disclosed in U.S. Ser. No. 09/475,393, filed Dec. 30, 1999, and entitled “Stent Designs For Use in Peripheral Vessels,” the contents of which are hereby incorporated by reference in its entirety. However, the radiopaque interconnecting elements may be configured for use with many other stent designs, including the ACCULINK™ stent also manufactured by Advanced Cardiovascular Systems, Inc., Santa Clara, Calif., among others.

As indicated in the table set forth below, the thickness and length approximations for the DYNALINK™ and ACCULINK™ stents, representative of Advanced Cardiovascular Systems, Inc.'s existing Nitinol stent patterns, can be used as a guide for making the radiopaque interconnecting elements 20 and 32 of the same parameters:

| Stent Pattern | Thickness (in) | Length (in) |
|---|---|---|
| Acculink | 0.0050 | 0.0575 |
| Dynalink 5-8 mm | 0.0075 | 0.0609 |
| Dynalink 9-10 mm | 0.0075 | 0.0715 |
| Dynalink 12-14 mm | 0.0110 | 0.1035 |

Based on the approximations in the above table, the plurality of radiopaque interconnecting elements 20 and 32 can have a thickness in the range of approximately 0.0050 to 0.0110 inch, and a length in the range of approximately 0.0575 to 0.1035 inch. It should be appreciated that these ranges are only representative for the types of stents indicated therein, and other types of stents may be used in combination with either variation of the radiopaque interconnecting elements 20 and 32.

The implantation of the stent of the present invention is readily apparent during a radiological examination of the patient later in time as the bright images generated by the radiopaque interconnecting elements cannot be overlooked. Additionally, because the radiopaque components are expanded against the vessel walls, they remain in place even if one or both end rings becomes separated from the rest of the stent. As a result, the position of the stent continues to be clearly discernable and the possibility of an end ring from migrating is effectively obviated.

The aforedescribed illustrative stent 10 of the present invention and similar stent structures can be made in many ways. One method of making the stent rings 11 is to cut a thin-walled tubular member, such as stainless steel tubing to remove portions of the tubing in the desired pattern for the stent, leaving relatively untouched the portions of the metallic tubing which are to form the rings. In accordance with the invention, it is preferred to cut the tubing in the desired pattern using a machine-controlled laser.

The tubing may be made of suitable biocompatible material such as stainless steel, cobalt-chromium (CoCn, NP35N), titanium, nickel-titanium (NiTi), tungsten, tantalum, and similar alloys. The stainless steel tube may be alloy type: 316L SS, Special Chemistry per ASTM F138-92 or ASTM F139-92 grade 2. Special Chemistry of type 316L per ASTM F138-92 or ASTM F139-92 Stainless Steel for Surgical Implants in weight percent.

| | |
|---|---|
| Carbon (C) | 0.03% max. |
| Manganese (Mn) | 2.00% max. |
| Phosphorous (P) | 0.025% max. |
| Sulphur (S) | 0.010% max. |
| Silicon (Si) | 0.75% max. |
| Chromium (Cr) | 17.00-19.00% |
| Nickel (Ni) | 13.00-15.50% |
| Molybdenum (Mo) | 2.00-3.00% |
| Nitrogen (N) | 0.10% max. |
| Copper (Cu) | 0.50% max. |
| Iron (Fe) | Balance |

The stent diameter is very small, so the tubing from which it is made must necessarily also have a small diameter. Typically the stent has an outer diameter on the order of about 0.06 inch in the unexpanded condition, the same outer diameter of the tubing from which it is made, and can be expanded to an outer diameter of 0.1 inch or more. The wall thickness of the tubing is about 0.003 inch.

The tubing is put in a rotatable collet fixture of a machine-controlled apparatus for positioning the tubing relative to a laser. According to machine-encoded instructions, the tubing is rotated and moved longitudinally relative to the laser which is also machine-controlled. The laser selectively removes the material from the tubing by ablation and a pattern is cut into the tube. The tube is therefore cut into the discrete pattern of the finished cylindrical rings.

Cutting a fine structure (0.0035 inch web width) requires minimal heat input and the ability to manipulate the tube with precision. It is also necessary to support the tube yet not allow the stent structure to distort during the cutting operation. In one embodiment, the tubes are made of stainless steel with an outside diameter of 0.060 inch to 0.095 inch and a wall thickness of 0.002 inch to 0.004 inch. These tubes are fixtured under a laser and positioned utilizing a CNC to generate a very intricate and precise pattern. Due to the thin wall and the small geometry of the stent pattern (0.0035 inch typical strut or ring width), it is necessary to have very precise control of the laser, its power level, the focused spot size, and the precise positioning of the laser cutting path.

In order to minimize the heat input into the stent structure, which prevents thermal distortion, uncontrolled bum out of the metal, and metallurgical damage due to excessive heat, and thereby produce a smooth debris free cut, a Q-switched Nd/YAG, typically available from Quantronix of Hauppauge, N.Y., that is frequency doubled to produce a green beam at 532 nanometers is utilized. Q-switching produces very short pulses (<100 nS) of high peak powers (kilowatts), low energy per pulse (≦3 mJ), at high pulse rates (up to 40 kHz). The frequency doubling of the beam from 1.06 microns to 0.532 microns allows the beam to be focused to a spot size that is 2 times smaller, therefore increasing the power density by a factor of 4 times. With all of these parameters, it is possible to make smooth, narrow cuts in the stainless tubes in very fine geometries without damaging the narrow struts that make up to stent structure. Hence, the system makes it possible to adjust the laser parameters to cut narrow kerf width which will minimize the heat input into the material.

The positioning of the tubular structure requires the use of precision CNC equipment such as that manufactured and sold by Anorad Corporation. In addition, a unique rotary mechanism has been provided that allows the computer program to be written as if the pattern were being cut from a flat sheet. This allows both circular and linear interpolation to be utilized in programming. Since the finished structure of the stent is very small, a precision drive mechanism is required that supports and drives both ends of the tubular structure as it is cut. Since both ends are driven, they must be aligned and precisely synchronized, otherwise the stent structure would twist and distort as it is being cut.

The optical system which expands the original laser beam, delivers the beam through a viewing head and focuses the beam onto the surface of the tube, incorporates a coaxial gas jet and nozzle that helps to remove debris from the kerf and cools the region where the beam interacts with the material as the beam cuts and vaporizes the metal. It is also necessary to block the beam as it cuts through the top surface of the tube and prevent the beam, along with the molten metal and debris from the cut, from impinging on the opposite surface of the tube.

In addition to the laser and the CNC positioning equipment, the optical delivery system includes a beam expander to increase the laser beam diameter, a circular polarizer, typically in the form of a quarter wave plate, to eliminate polarization effects in metal cutting, provisions for a spatial filter, a binocular viewing head and focusing lens, and a coaxial gas jet that provides for the introduction of a gas stream that surrounds the focused beam and is directed along the beam axis. The coaxial gas jet nozzle (0.018 inch I.D.) is centered around the focused beam with approximately 0.010 inch between the tip of the nozzle and the tubing. The jet is pressurized with oxygen at 20 psi and is directed at the tube with the focused laser beam exiting the tip of the nozzle (0.018 inch dia.) The oxygen reacts with the metal to assist in the cutting process very similar to oxyacetylene cutting. The focused laser beam acts as an ignition source and controls the reaction of the oxygen with the metal. In this manner, it is possible to cut the material with a very fine kerf with precision. In order to prevent burning by the beam and/or molten slag on the far wall of the tube I.D., a stainless steel mandrel (approx. 0.034 inch dia.) is placed inside the tube and is allowed to roll on the bottom of the tube as the pattern is cut. This acts as a beam/debris block protecting the far wall I.D.

Alternatively, this may be accomplished by inserting a second tube inside the stent tube which has an opening to trap the excess energy in the beam which is transmitted through the kerf along which collecting the debris that is ejected from the laser cut kerf. A vacuum or positive pressure can be placed in this shielding tube to remove the collection of debris.

Another technique that could be utilized to remove the debris from the kerf and cool the surrounding material would be to use the inner beam blocking tube as an internal gas jet. By sealing one end of the tube and making a small hole in the side and placing it directly under the focused laser beam, gas pressure could be applied creating a small jet that would force the debris out of the laser cut kerf from the inside out. This would eliminate any debris from forming or collecting on the inside of the stent structure. It would place all the debris on the outside. With the use of special protective coatings, the resultant debris can be easily removed.

In most cases, the gas utilized in the jets may be reactive or non-reactive (inert). In the case of reactive gas, oxygen or compressed air is used. Compressed air is used in this application since it offers more control of the material removed and reduces the thermal effects of the material itself. Inert gas such as argon, helium, or nitrogen can be used to eliminate any oxidation of the cut material. The result is a cut edge with no oxidation, but there is usually a tail of molten material that collects along the exit side of the gas jet that must be mechanically or chemically removed after the cutting operation.

The cutting process utilizing oxygen with the finely focused green beam results in a very narrow kerf (approx. 0.0005 inch) with the molten slag re-solidifying along the cut. This traps the cut out scrap of the pattern requiring further processing. In order to remove the slag debris from the cut allowing the scrap to be removed from the remaining stent pattern, it is necessary to soak the cut tube in a solution of HCl for approximately 8 minutes at a temperature of approximately 55° C. Before it is soaked, the tube is placed in a bath of alcohol/water solution and ultrasonically cleaned for approximately 1 minute to remove the loose debris left from the cutting operation. After soaking, the tube is then ultrasonically cleaned in the heated HCl for 1-4 minutes depending upon the wall thickness. To prevent cracking/breaking of the struts attached to the material left at the two ends of the stent pattern due to harmonic oscillations induced by the ultrasonic cleaner, a mandrel is placed down the center of the tube during the cleaning/scrap removal process. At completion of this process, the stent structures are rinsed in water. They are now ready for electropolishing.

The stent rings are preferably electrochemically polished in an acidic aqueous solution such as a solution of ELECTRO-GLO #300, sold by the ELECTRO-GLO Co., Inc. in Chicago, Ill., which is a mixture of sulfuric acid, carboxylic acids, phosphates, corrosion inhibitors and a biodegradable surface active agent. The bath temperature is maintained at about 110-135° F. and the current density is about 0.4 to about 1.5 amps per in.² Cathode to anode area should be at least about two to one.

Direct laser cutting produces edges which are essentially perpendicular to the axis of the laser cutting beam, in contrast with chemical etching and the like which produce pattern edges which are angled. Hence, the laser cutting process essentially provides strut cross-sections, from cut-to-cut, which are square or rectangular, rather than trapezoidal.

The foregoing laser cutting process to form the cylindrical rings 11 can be used with other metals including cobalt-chromium, titanium, tantalum, nickel-titanium, and other biocompatible metals suitable for use in humans, and typically used for intravascular stents. Further, while the formation of the cylindrical rings is described in detail, other processes of forming the rings are possible and are known in the art, such as by using chemical etching, electronic discharge machining, stamping, and other processes.

While the invention has been described in connection with certain disclosed embodiments, it is not intended to limit the scope of the invention to the particular forms set forth, but, on the contrary it is intended to cover all such alternatives, modifications, and equivalents as may be included in the spirit and scope of the invention as defined by the appended claims.

What is claimed:

1. A longitudinally flexible stent having high visibility under fluoroscopy for implanting in a body lumen, comprising:
a plurality of adjacent cylindrical elements, each cylindrical element having a circumference extending about a longitudinal stent axis and being substantially independently expandable in the radial direction;
wherein the plurality of adjacent cylindrical elements are arranged in alignment along the longitudinal stent axis and form a generally serpentine wave pattern transverse to the longitudinal axis containing a plurality of alternating valley portions and peak portions;
a plurality of interconnecting elements extending between the adjacent cylindrical elements and connecting the adjacent cylindrical elements to one another;
a plurality of radiopaque interconnecting elements configured to be expandable while encapsulated by polymeric material;
wherein the plurality of radiopaque interconnecting elements are connected to the cylindrical elements at least at distal and proximal ends of the stent so that the radiopaque interconnecting elements are visible under fluoroscopy and the distal and proximal stent ends can be easily located in the body lumen at the implantation site.

2. The stent of claim 1, wherein a radiopaque portion of the plurality of radiopaque interconnecting elements is shaped in a coiled configuration.

3. The stent of claim 2, wherein the coil of the radiopaque interconnecting elements is formed from at least one of a rectangular flatwire and a round wire.

4. The stent of claim 2, wherein the coil of the radiopaque interconnecting elements has a cross-sectional area and a pitch that can be changed to modify performance properties of the coil.

5. The stent of claim 4, wherein an increase in at least one of a width and a thickness of the flatwire coil adds strength and rigidity to the coil.

6. The stent of claim 4, wherein an increase in a diameter of the round wire coil adds strength and rigidity to the coil.

7. The stent of claim 4, wherein an increase in the pitch of the coil adds flexibility to the coil.

8. The stent of claim 1, wherein the radiopaque interconnecting elements comprise a metal selected from the group of metals consisting of gold, platinum, tantalum, and platinum/10% iridium.

9. The stent of claim 1, wherein the plurality of radiopaque interconnecting elements have a thickness in the range of approximately 0.0050 to 0.0110 inch, and a length in the range of approximately 0.0575 to 0.1035 inch.

10. The stent of claim 1, wherein the polymeric material encapsulating the plurality of radiopaque interconnecting elements comprises a biocompatible polymer.

11. The stent of claim 10, wherein the biocompatible polymer is urethane.

12. The stent of claim 1, wherein the plurality of radiopaque interconnecting elements are connected to the cylindrical elements throughout the length of the stent so that the radiopaque interconnecting elements are visible under fluoroscopy and the stent can be easily located in the body lumen at the implantation site.

13. The stent of claim 1, wherein the plurality of radiopaque interconnecting elements are selectively placed on at least one low stress area of the stent.

14. The stent of claim 13, wherein the at least one low stress area includes a strut member of the plurality of adjacent cylindrical elements.

15. The stent of claim 1, wherein the plurality of radiopaque interconnecting elements encapsulated by polymeric material are attached to at least the distal and proximal ends of the stent by micro-injection molding.

16. The stent of claim 1, wherein the plurality of radiopaque interconnecting elements encapsulated by polymeric material are attached to at least the distal and proximal ends of the stent by micro-welding.

17. The stent of claim 1, wherein the plurality of radiopaque interconnecting elements encapsulated by polymeric material are attached to at least the distal and proximal ends of the stent by resistance welding.

18. The stent of claim 1, wherein the plurality of radiopaque interconnecting elements encapsulated by polymeric material are attached to at least the distal and proximal ends of the stent by a mechanical junction.

19. The stent of claim 1, wherein the plurality of radiopaque interconnecting elements alternate with a plurality of non-radiopaque elements at the distal and proximal ends of the stent.

20. The stent of claim 1, wherein the plurality of cylindrical elements are formed from a tubular member.

21. The stent of claim 1, wherein the plurality of cylindrical elements are formed from a flat sheet of material.

22. The stent of claim 1, wherein the stent includes a pseudoelastic alloy material.

23. The stent of claim 1, wherein the stent includes a shape memory alloy material.

24. The stent of claim 1, wherein the stent is formed from a biocompatible material selected from the group consisting of stainless steel, tungsten, tantalum, superelastic nickel titanium alloys, and thermal plastic polymers.

25. A longitudinally flexible stent having high visibility under fluoroscopy for implanting in a body lumen, comprising:
a plurality of adjacent cylindrical elements, each cylindrical element having a circumference extending about a longitudinal stent axis and being substantially independently expandable in the radial direction;
wherein the plurality of adjacent cylindrical elements are arranged in alignment along the longitudinal stent axis and form a generally serpentine wave pattern transverse to the longitudinal axis containing a plurality of alternating valley portions and peak portions;
a plurality of interconnecting elements extending between the adjacent cylindrical elements and connecting the adjacent cylindrical elements to one another; and
a plurality of radiopaque interconnecting elements configured to be expandable while encapsulated by polymeric material, wherein the radiopaque interconnecting elements are connected to the cylindrical elements of the stent so that the radiopaque interconnecting elements are visible under fluoroscopy and the stent can be easily located in the body lumen at the implantation site.

26. A longitudinally flexible stent having high visibility under fluoroscopy for implanting in a body lumen, comprising:
a plurality of adjacent cylindrical elements, each cylindrical element having a circumference extending about a longitudinal stent axis and being substantially independently expandable in the radial direction;
wherein the plurality of adjacent cylindrical elements are arranged in alignment along the longitudinal stent axis and form a generally serpentine wave pattern transverse to the longitudinal axis containing a plurality of alternating valley portions and peak portions;
a plurality of interconnecting elements extending between the adjacent cylindrical elements and connecting the adjacent cylindrical elements to one another; and
a plurality of radiopaque interconnecting elements having a polymeric material filled with a radiopaque material attached to the cylindrical elements of the stent, the plurality of radiopaque interconnecting elements connected to the cylindrical elements at least at distal and proximal ends of the stent so that the radiopaque material is visible under fluoroscopy and the distal and proximal stent ends can be easily located in the body lumen at the implantation site.

27. The stent of claim 26, wherein the plurality of radiopaque interconnecting elements are configured to assume a linear configuration.

28. The stent of claim 26, wherein the plurality of radiopaque interconnecting elements are configured to assume a non-linear configuration.

29. The stent of claim 26, wherein the plurality of radiopaque interconnecting elements are configured to assume a VTS configuration.

30. The stent of claim 26, wherein the plurality of cylindrical elements are formed from a tubular member.

31. The stent of claim 26, wherein the plurality of cylindrical elements are formed from a flat sheet of material.

32. The stent of claim 26, wherein the stent includes a pseudoelastic alloy material.

33. The stent of claim 26, wherein the stent includes a shape memory alloy material.

34. The stent of claim 26, wherein the stent is formed from a biocompatible material selected from the group consisting of stainless steel, tungsten, tantalum, superelastic nickel titanium alloys, and thermal plastic polymers.

35. The stent of claim 26, wherein the polymeric material comprises a biocompatible polymer.

36. The stent of claim 35, wherein the biocompatible polymer is urethane.

37. The stent of claim 26, wherein the radiopaque material is selected from the group of materials consisting of tungsten, and barium sulfate.

38. The stent of claim 26, wherein the plurality of radiopaque interconnecting elements have a thickness in the range of approximately 0.0050 to 0.0110 inch, and a length in the range of approximately 0.0575 to 0.1035 inch.

39. The stent of claim 26, wherein the radiopaque polymeric material is in communication with the interconnecting element to form the radiopaque interconnecting element.

40. The stent of claim 39, wherein the radiopaque polymeric material is attached to the interconnecting element by micro-injection molding.

41. The stent of claim 26, wherein the plurality of radiopaque interconnecting elements are connected to adjacent cylindrical elements throughout the entire length of the stent.
